(19) United States
(12) Patent Application Publication
(10) Pub. No.: US 2024/0377344 A1
(43) Pub. Date: Nov. 14, 2024

(54) METHOD FOR CORRECTING AGE OF COLUMBITE-TANTALITE BY USING DOUBLE-REFERENCE-MATERIALS

(71) Applicant: INSTITUTE OF GEOLOGY AND GEOPHYSICS CHINESE ACADEMY OF SCIENCES, Beijing (CN)

(72) Inventors: Xiaoxiao LING, Beijing (CN); Yu LIU, Beijing (CN); Xianhua LI, Beijing (CN); Hongxia MA, Beijing (CN)

(73) Assignee: INSTITUTE OF GEOLOGY AND GEOPHYSICS CHINESE ACADEMY OF SCIENCES, Beijing (CN)

(21) Appl. No.: 18/643,765

(22) Filed: Apr. 23, 2024

(30) Foreign Application Priority Data
May 5, 2023 (CN) 202310493629.3

(51) Int. Cl. G01N 23/2258; G01N 33/24; H01J 49/00; H01J 49/04; H01J 49/14

(52) U.S. Cl. CPC G01N 23/2258; G01N 33/24; H01J 49/0031; H01J 49/0036; H01J 49/0409; H01J 49/142

(57) ABSTRACT

A method for accurately correcting the age of columbite-tantalite by using double-reference-material includes: using at least two kinds of columbite-tantalite with known ages as reference materials; determining the positive correlation between the lead-uranium ion ratio and the uranium ion ratio of the columbite-tantalite based on test results of a lead-uranium ion ratio

$${( \frac{{206}^{} {Pb}^{+}}{{238}^{} {U}^{16} {O}^{+}} )}_{Cstd 1}$$

and a uranium ion ratio

$${( \frac{{238}^{} {U}^{16} {O}_{2}^{+}}{{238}^{} {U}^{16} {O}^{+}} )}_{Cstd 1}$$

of the reference material 1, correcting the correlation by reference material 2; calculating the lead-uranium mass ratio

$${( \frac{{206}^{} Pb}{{238}^{} U} )}_{un}$$

of the columbite-tantalite sample to be tested by using the corrected correlation; finally, determining the uranium-lead age UPbtᵤₙ of the columbite-tantalite sample to be tested based on the lead-uranium mass ratio

$${( \frac{{206}^{} Pb}{{238}^{} U} )}_{un} .$$

The method is fast and reliable, and greatly increases the accuracy in calculating the age of columbite-tantalite, and the method represents the latest international test design and calibration method in this field, and is of great practical value.

### TECHNICAL FIELD

The present disclosure relates to the field of age determination for columbite-tantalite, and in particular relates to a method for high-accuracy age determination of micron-scale columbite-tantalite.

### BACKGROUND

Radioisotope dating is a method to obtain an absolute age in geological researches. Accurate isotope chronology is of great significance in reversing geological historical events and exploring the geodynamic background of diagenesis and mineralization and the genesis of mineral deposits, and, in particular, is crucial for many studies on metamorphic rocks and hydrothermal deposits with unconspicuous chronological order of multi-stage hydrothermal events. The basic method is to accurately measure the content ratio of daughter radioisotope to the remaining parent radioisotope in the geological body, and then calculate the elapsed time experienced by the geological body according to the half-life principle. At present, uranium-lead (U—Pb) dating, due to its appropriate characteristics, is the most widely used method in the study of chronology for solid earth science. Current accessory minerals used for dating mainly include zircon, monazite, titanite, bastnaesite, columbite-tantalite and the like.

Columbite-tantalite [(Fe, Mn)(Nb, Ta)₂O₆], the main ore mineral of key resources niobium and tantalum (Reference 1: McCaffrey et al., 2023), is mainly hosted in rare metal granites, alkaline and carbonate rocks, pegmatites, and hydrothermal veins. The dating of columbite-tantalite facilitates exploration and discovery of niobium and tantalum resources. Due to the high content of U and Th, the columbite-tantalite is a mineral suitable for dating. However, many columbite-tantalite minerals show the characteristics of multi-stage growth in rocks and small particle size (generally 5-50 microns, mainly about 10 microns). It is necessary to use a high-accuracy dating method to directly determine the age of columbite-tantalite in rocks, so as to solve the problem of age determination for columbite-tantalite growing in different stages.

As reported in the international literatures, the methods for accurate dating of columbite-tantalite include isotope dilution thermo-ionization mass spectrometry (ID-TIMS) (Reference 2: Smith et al., 2004), laser ablation inductively coupled plasma mass spectrometry (LA-ICP-MS) (Reference 3: Zhao et al., 2021), and secondary ion mass spectrometry (SIMS) (Reference 4: Legros et al., 2019). These three dating methods have their own advantages and disadvantages.

ID-TIMS is the most accurate method for dating, which involves breaking rock samples, picking the columbite-tantalite therefrom, dissolving the columbite-tantalite into a solution with acid at a high temperature, then measuring the contents of uranium and lead in the solution, and calculating the age. At present, there are many geological applications involving the use of ID-TIMS for U—Pb dating of the columbite-tantalite, but the high-accuracy ID-TIMS is time-consuming. Therefore, in-situ dating techniques such as LA-ICP-MS and SIMS have emerged with small sampling volume and short test cycle.

LA-ICP-MS eliminates the need for mineral picking, and allows analysis by cutting a rock into thin sections and directly selecting a columbite-tantalite grain. However, this analysis method is generally used to measure particles with a diameter of more than 30 microns, and thus cannot accurately determine the age of columbite-tantalite due to the small size of columbite-tantalite in most rocks.

SIMS is to perform in-situ microanalysis on the columbite-tantalite, showing the characteristics of low sample consumption, availability for long-term analysis, repeatability and the like. Due to many cracks in the columbite-tantalite, an ion probe with high accuracy and high-spatial resolution is used for in-situ microanalysis of the columbite-tantalite, such that the problem of inclusions can be effectively avoided to achieve more accurate results.

However, SIMS is rarely used at present to study the columbite-tantalite because of matrix effects which are one of the main issues to be considered in the in situ microanalysis of SIMS. Legros et al. (2019) has established a method for U—Pb dating of columbite-tantalite by an ion probe, in which an electron probe (EPMA) is needed for matrix effect correction and several columbite-tantalite reference materials with homogeneous compositions are recommended. However, this method needs to be improved because the analysis areas of electron probes and ion probes do not overlap completely and multiple analysis is time-consuming and laborious.

Therefore, the present disclosure designs a novel method for in-situ determination of micron-scale columbite-tantalite, in which two reference materials are used to navigate the correction to obtain an accurate age. This method can not only save time and labor, but also is precise and accurate for solving the unsolved problem.

### REFERENCES

Reference 1: McCaffrey, D. M., Nassar, N. T., Jowitt, S. M., Padilla, A. J., Bird, L. R., 2023. Embedded critical material flow: The case of niobium, the United States, and China. Resources, Conservation and Recycling, 188.
Reference 2: Smith, S. R. et al., 2004. U—Pb columbite-tantalite chronology of rare-element pegmatites using TIMS and Laser Ablation-Multi Collector-ICP-MS. Contributions to Mineralogy and Petrology, 147(5): 549-564.
Reference 3: Zhao Junxing, He Changtong, Qin Kezhang et al., Geochronology, source features and the characteristics of fractional crystallization in pegmatite at the Qongjiagang giant pegmatite-type lithium deposit, Himalaya, XiZang [J]. Acta Petrologica Sinica, 2021, 37(11): 3325-3347.
Reference 4: Legros, H. et al., 2019. U—Pb isotopic dating of columbite-tantalite minerals: Development of reference materials and in situ applications by ion microprobe. Chemical Geology, 512: 69-84.

### SUMMARY

All references mentioned in the present disclosure are incorporated herein by reference. Unless otherwise indicated, all technical and scientific terms used herein have the same meanings as generally understood by those of ordinary skill in the art to which the present disclosure belongs. Unless otherwise indicated, the techniques used or mentioned herein are standard techniques generally known to those of ordinary skill in the art. The materials, methods and examples are for illustrative purposes, rather than limiting.

In the field of age determination of columbite-tantalite, the methods already published at present require the use of an electron probe to measure the element content of a sample to obtain composition data, and the subsequent use of an ion probe to determine the isotope ratio of columbite-tantalite. A result can only be obtained by combining the data produced by the two probes, and this method needs to be improved since it not only requires multiple measurements by the two probes, which is time-consuming and laborious, but also brings deviations and increases errors due to multiple measurements.

An object of the present disclosure is to overcome the defects of the prior art and provide a method for high-accuracy age determination of micron-scale columbite-tantalite. Through a large number of experiments and comparative analysis, the inventor of the present disclosure has found an ion pair for accurately correcting the lead-uranium age of columbite-tantalite, and established a corresponding calculation method.

Optionally, the embodiments of the present disclosure provides a method for high-accuracy age determination of micron-scale columbite-tantalite. With at least two kinds of columbite-tantalite with known ages as reference materials, the method disclosed in the present disclosure involves the following procedures: positive correlation between the lead-uranium ion ratio and the uranium ion ratio of the columbite-tantalite is determined based on testing results of a lead-uranium ion ratio

$${( \frac{{206}^{} {Pb}^{+}}{{238}^{} {U}^{16} {O}^{+}} )}_{Cstd 1}$$

and a uranium ion ratio

$${( \frac{{238}^{} {U}^{16} {O}_{2}^{+}}{{238}^{} {U}^{16} {O}^{+}} )}_{Cstd 1}$$

of a reference material 1, and the correlation is corrected by the reference material 2, and the lead-uranium mass ratio

$${( \frac{{206}^{} Pb}{{238}^{} U} )}_{un}$$

of columbite-tantalite is calculated by using the corrected correlation; finally, the uranium-lead age UPbtᵤₙ of columbite-tantalite sample to bested is determined based on the lead-uranium mass ratio

$${( \frac{{206}^{} Pb}{{238}^{} U} )}_{un} .$$

Optionally, an embodiment of the present disclosure provides a method for high-accuracy age determination of micron-scale columbite-tantalite, comprising, using two kinds of columbite-tantalite with known ages and different compositions as reference materials; determining the positive correlation between the lead-uranium ion ratio

$${( \frac{{206}^{} {Pb}^{+}}{{238}^{} {U}^{16} {O}^{+}} )}_{CStd 1}$$

and the uranium ion ratio

$${( \frac{{238}^{} {U}^{16} {O}_{2}^{+}}{{238}^{} {U}^{16} {O}^{+}} )}_{Cstd 1}$$

of the reference material 1; calculating the lead-uranium isotope ratio

$${( \frac{{206}^{} Pb}{{238}^{} U} )}_{Cstd 2}$$

and the lead-uranium age UPbt_{Cstd2} of the reference material 2 based on the same positive correlation; calculating the lead-uranium ratio

$${( \frac{{206}^{} Pb}{{238}^{} U} )}_{Cun}$$

and the lead-uranium age UPbt_{Cun} of the sample to be tested based on the determined positive correlation and the lead-uranium iron ratio

$${( \frac{{206}^{} {Pb}^{+}}{{238}^{} {U}^{16} {O}^{+}} )}_{Cun}$$

and the uranium iron ratio

$${( \frac{{238}^{} {U}^{16} {O}_{2}^{+}}{{238}^{} {U}^{16} {O}^{+}} )}_{Cun}$$

of the sample to be tested.

Optionally, wherein the method further comprising: after obtaining the lead-uranium age UPbt_{std2} of the reference material 2, the method further includes performing deviation correction, determining a positive correlation after correction, and then calculating the lead-uranium ratio

$${( \frac{{206}^{} Pb}{{238}^{} U} )}_{Cun}$$

and the lead-uranium age UPbt_{Cun} of the sample to be tested.

Optionally, wherein the method further comprises:
S1, measuring the reference material 1 to obtain not less than 3 sets of the lead-uranium ion ratio

$${( \frac{{206}^{} {Pb}^{+}}{{238}^{} {U}^{16} {O}^{+}} )}_{Cstd 1}$$

and the uranium ion ratio

$${( \frac{{238}^{} {U}^{16} {O}_{2}^{+}}{{238}^{} {U}^{16} {O}^{+}} )}_{Cstd 1} ,$$

and performing fitting according to the equation

$$ln ( \frac{{206}^{} {Pb}^{+}}{{238}^{} {U}^{16} {O}^{+}} ) = B \times ln ( \frac{{238}^{} {U}^{16} {O}_{2}^{+}}{{238}^{} {U}^{16} {O}^{+}} ) + ln ( A )$$

to obtain the values of A and B;
S2, measuring the reference material 2 to obtain the lead-uranium ion ratio

$${( \frac{{206}^{} {Pb}^{+}}{{238}^{} {U}^{16} {O}^{+}} )}_{Cstd 2}$$

and the uranium ion ratio

$${( \frac{{238}^{} {U}^{16} {O}_{2}^{+}}{{238}^{} {U}^{16} {O}^{+}} )}_{Cstd 2}$$

of the reference material 2, substituting the values of A and B from S1 and the recommended value

$${( \frac{{206}^{} Pb}{{238}^{} U} )}_{Cstd 1}$$

of the reference material 1 into the equation

$${( \frac{{206}^{} Pb}{{238}^{} U} )}_{Cstd 2} = {( \frac{{206}^{} Pb}{{238}^{} U} )}_{Cstd 1} \times \frac{{( \frac{{206}^{} {Pb}^{+}}{{238}^{} {U}^{16} {O}^{+}} )}_{Cstd 2}}{A \times {( \frac{{238}^{} {U}^{16} {O}_{2}^{+}}{{238}^{} {U}^{16} {O}^{+}} )}_{Cstd 2}^{B}} ,$$

calculate the corrected lead-uranium ratio

$${( \frac{{206}^{} Pb}{{238}^{} U} )}_{Cstd 2}$$

of the reference material 2, and calculating measured age UPbt_{Cstd2} of the reference material 2 according to the equation:

$$UPbt = ln ( ( \frac{{206}^{} Pb}{{238}^{} U} ) + 1 ) / {λ}_{238} ,$$

λ₂₃₈ is a decay constant, and λ₂₃₈=1.55125×10⁻¹⁰;
S3, calculating the deviation value of the recommended age from the measured age of the reference material 2 according to the equation Δt=Age_{UPbCStd2}−UPbt_{Cstd2};
S4, if a value of Δt is greater than Age_{UPbCStd2}×deviation of the reference material 2, recalculating the value of B with the above B=0.1 as a starting value of iteration, B=5 as an end value, and a step of 1%, substituting each value of B obtained into S2 and S3 to determine a value of B when Δt is less than or equal to the Age_{UPbCStd2}×deviation of the reference material 2, and if Δt is less than a value obtained by the Age_{UPbCStd2}×deviation of the reference material 2, directly going to S5; and
S5, measuring the lead-uranium ion ratio

$${( \frac{{206}^{} {Pb}^{+}}{{238}^{} {U}^{16} {O}^{+}} )}_{Cun}$$

and the uranium ion ratio

$${( \frac{{238}^{} {U}^{16} {O}_{2}^{+}}{{238}^{} {U}^{16} {O}^{+}} )}_{Cun}$$

of the sample to be tested, substituting the recommended value

$${( \frac{{206}^{} Pb}{{238}^{} U} )}_{Cstd 1}$$

of the reference material 1 Cstd1, the value of A, and the value of B obtained when Δt is less than or equal to the Age_{UPbCStd2}×deviation of the reference material 2 into the equation

$${( \frac{{206}^{} Pb}{{238}^{} U} )}_{Cun} = {( \frac{{206}^{} Pb}{{238}^{} U} )}_{Cstd 1} \times \frac{{( \frac{{206}^{} {Pb}^{+}}{{238}^{} {U}^{16} {O}^{+}} )}_{Cun}}{A \times {( \frac{{238}^{} {U}^{16} {O}_{2}^{+}}{{238}^{} {U}^{16} {O}^{+}} )}_{Cun}^{B}} ,$$

calculating the corrected lead-uranium ratio

$${( \frac{{}^{206} Pb}{{}^{238} U} )}_{C u n}$$

of the columbite-tantalite sample Cun, and calculate the age value UPbt_{Cun} of the sample to be tested according to the equation

$$UPbt = ln ( ( \frac{{}^{206} Pb}{{}^{238} U} ) + 1 ) / {λ}_{238} .$$

Optionally, wherein the two reference materials provided in the embodiment of the present disclosure may be samples of any age and composition. Preferably, the reference materials are a columbite-tantalite reference material CStd1 (Nb₂O₅≈55.1%, Ta₂O₅β24.2%, FeO≈13.0%, MnO≈57%, TiO₂≈0.5%, UPbt_{CStd1}≈380 Ma (million years)) and a columbite-tantalite reference material CStd2 (Nb₂O₅≈48.0%, Ta₂O₅≈32.4%, FeO≈6.0%, MnO≈12.3%, TiO₂≈0.1%, UPbt_{CStd2}≈264 Ma (million years)).

Optionally, before the samples are measured, the method further includes the steps of:
step 1, preparing a combined sample mount from the two reference materials and a columbite-tantalite sample to be tested;
step 2, washing the sample mount; and
step 3, plating the sample mount with a conductive material.

Optionally, preparing the combined sample mount includes the steps of:
step 1, applying a double-sided adhesive tape of 10 cm*5 cm to a glass sheet of 10 cm*10 cm, and adhering 7-10 particles of each of the reference material 1, the reference material 2, and the sample Cun1 with a particle size of 100-150 microns respectively onto the double-sided adhesive tape within a circle with a diameter of about 2 mm; and
step 2, placing a hollow polyethylene column with a smooth surface vertically on the double-sided adhesive tape, slowing pouring a vacuumized mixture of epoxy resin and a coagulant along an inner surface of the hollow polyethylene column, performing vacuumization again and allowing standing to coagulate the mixture to obtain a coagulated columbite-tantalite reference material grains, i.e., a columbite-tantalite mount, that can be taken out from the hollow polyethylene column, and polishing the columbite-tantalite mount by sequentially using fine sandpaper and a polishing disk to expose the reference materials and the sample from a side of the mount.

Optionally, plating the sample mount with the conductive material includes plating a surface of a cleaned disc at a side with exposed samples, with a continuous gold coating with a thickness of 20 nm-50 nm.

Optionally, measuring includes measuring ion signals of the columbite-tantalite by using a secondary ion mass spectrometer SIMS.

Optionally, wherein measuring the ion signals of the columbite-tantalite by using the secondary ion mass spectrometer (SIMS) includes the steps of:
step 1, first, focusing an oxygen plasma ion source onto the columbite-tantalite samples on the sample mount by means of Gaussian light to produce secondary ions of the columbite-tantalite samples; and
step 2, allowing secondary ions ²⁰⁴Pb⁺, ²⁰⁶Pb⁺, ²⁰⁷Pb⁺, ¹⁸¹Ta¹⁸O¹⁶O⁺, ¹⁸⁴W¹⁶O₂⁺, ²³⁸U⁺, ²³⁸U¹⁶O⁺, and ²³⁸U¹⁶O₂⁺ of the samples to sequentially pass through an electric field and a magnetic field to reach an ion signal detection system, and performing detection to obtain a signal intensity of each of the secondary ions.

The present disclosure has the following advantages:
(1) the uranium-lead ion type of the columbite-tantalite can be accurately measured, and the age of the sample can be accurately calculated through the using of the two reference materials; and
(2) the method provided by the present disclosure is fast and reliable, and greatly increases the accuracy in calculating the age of columbite-tantalite, and the method is the up-to-date test design and correction method across the world in the art at present, showing great application value in fields such as geology.

### BRIEF DESCRIPTION OF THE DRAWINGS

FIG. 1 shows a schematic diagram of a combined sample mount containing two reference materials and a sample to be tested according to an embodiment of the present disclosure, with CStd1 indicating a reference material 1, CStd2 indicating a reference material 2, and Cun indicating a sample to be tested;

FIG. 2 shows positive correlation curves between the lead-uranium ion ratio

$$( \frac{{}^{206} {Pb}^{+}}{{}^{238} {U}^{16} {O}^{+}} )$$

and the uranium ion ratio

$$( \frac{{}^{238} {U}^{16} {O}_{2}^{+}}{{}^{238} {U}^{16} {O}^{+}} )$$

of the reference materials and the sample to be tested according to an embodiment of the present disclosure;

FIG. 3 shows a calculation flow chart according to an embodiment of the present disclosure; and

FIGS. 4A, 4B, and 4C show diagrams of the results of U—Pb age corrected for columbite-tantalite with different ion pairs, with a center point indicating an arithmetic mean of 20 analyses and the length of error bar indicating 1 sigma, FIG. 4A represents Cstd1, which is a columbite-tantalite reference material 1; FIG. 4B represents Cstd2, which is a columbite-tantalite reference material 2; and FIG. 4C represents Cun, which is an unknown columbite-tantalite sample to be tested.

### DETAILED DESCRIPTION OF THE EMBODIMENTS

To facilitate understanding of the present disclosure, the following provides a more comprehensive description of the present disclosure. However, the present disclosure may be implemented in many different forms and is not limited to the embodiments described herein. On the contrary, these embodiments are provided for the purpose of more thorough and comprehensive understanding of the disclosure of the present disclosure. It should be noted that the technical features or the combinations thereof described in the following embodiments should not be considered to be isolated, but may be combined with each other to achieve better technical effects.

In the present disclosure, unknown (abbreviated as un) and standard (abbreviated as std) are used to distinguish between the sample to be tested and the reference material, and they are marked at the bottom right of the ratio. The symbols, equations, and meanings involved in the present disclosure are shown in Table 1 below:

TABLE 1

| | Name, | Remark |
|---|---|---|
| Symbol | and meaning | |
| CStd1 | Columbite-tantalite reference material 1, | |
| | C is the initial of columbite-tantalite, and Std is | |
| | the abbreviation of standard, the same below. | |
| CStd2 | Columbite-tantalite reference material 2, | |
| Cun | Unknown columbite-tantalite sample to be tested, | |
| | and un is the abbreviation of unknown. | |
| Age_{UPbCStd1} | Recommended value of uranium-lead age for | |
| | columbite-tantalite reference material 1 | |
| Age_{UPbCStd2} | Recommended value of uranium-lead age for | |
| | columbite-tantalite reference material 2 | |
| UPbt, UPbt_{std}, UPbt_{cstd1}, UPbt_{cstd2}, UPbtᵤₙ, | The uranium-lead age of the sample calculated | |
| UPbt_{Cun} | using the method of the present disclosure, and | |
| | the subscripts Cstd1, Cstd2, and Cun represent the | |
| | columbite-tantalite reference material 1, the | |
| | columbite-tantalite reference material 2, and the | |
| | unknown columbite-tantalite sample to be tested, | |
| | respectively. The absence of subscript means no | |
| | distinction between the unknown sample to be | |
| | tested and the reference materials, the same below. | |
| $( \frac{{}^{206} P b}{{}^{238} U} ) , {( \frac{{}^{206} Pb}{{}^{238} U} )}_{Cstd 1} , {( \frac{{}^{206} Pb}{{}^{238} U} )}_{Cstd 1} , {( \frac{{}^{206} Pb}{{}^{238} U} )}_{C u n}$ | Lead-uranium mass ratio of columbite-tantalite | |
| $\begin{matrix}\frac{{}^{206} {Pb}^{+}}{{}^{238} {U}^{16} {O}^{+}} \\ {( \frac{{}^{206} {Pb}^{+}}{{}^{238} {U}^{16} {O}^{+}} )}_{Cstd 1} , {( \frac{{}^{206} {Pb}^{+}}{{}^{238} {U}^{16} {O}^{+}} )}_{Cstd 1} , {( \frac{{}^{206} {Pb}^{+}}{{}^{238} {U}^{16} {O}^{+}} )}_{C u n}\end{matrix}$ | Lead-uranium ion ratio, which is the directly measured lead-uranium ion ratio of columbite-tantalite. ²⁰⁶Pb⁺ indicates a lead ion with a mass of 206. ²³⁸U¹⁶O⁺ indicates a diatomic ion with a mass of 254, consisting of a uranium atom with a mass of 238 and an oxygen atom with a mass of 16. | |
| $\frac{{}^{238} {U}^{16} {O}_{2}^{+}}{{}^{238} {U}^{16} {O}^{+}} , {( \frac{{}^{238} {U}^{16} {O}_{2}^{+}}{{}^{238} {U}^{16} {O}^{+}} )}_{Cstd 1} , {( \frac{{}^{238} {U}^{16} {O}_{2}^{+}}{{}^{238} {U}^{16} {O}^{+}} )}_{Cstd 2} , {( \frac{{}^{238} {U}^{16} {O}_{2}^{+}}{{}^{238} {U}^{16} {O}^{+}} )}_{Cun}$ | Uranium ion ratio, which is the directly measured uranium ion ratio of columbite-tantalite. ²³⁸U¹⁶O₂⁺ indicates a uranium-containing ion with | |
| | a mass of 270, which is a three-atom molecular | |
| | ion consisting of a uranium atom with a mass | |
| | of 238 and two oxygen atoms each with a mass | |
| | of 16. | |
| $( \frac{{}^{206} P b}{{}^{238} U} ) = {e}^{{λ}_{238} * UPbt} - 1$ | Equation (1), which is a relational expression between the lead-uranium mass ratio and the uranium-lead age of the columbite-tantalite. λ₂₃₈ is the decay constant (1.55125 × 10⁻¹⁰) | Half-life principle |
| $UPbt = ln ( ( \frac{{}^{206} P b}{{}^{238} U} ) + 1 ) / {λ}_{238} \qquad ( 6 )$ | Equation (2), Age result Transformed from Equation (1) | Transformation |
| $\frac{{}^{206} {Pb}^{+}}{{}^{238} {U}^{16} {O}^{+}} = A \times {( \frac{{}^{238} {U}^{16} {O}_{2}^{+}}{{}^{238} {U}^{16} {O}^{+}} )}^{B}$ | Equation (3), Note 1: Both the unknown sample to be tested and the reference material conform to this equation. Note 2: A and B are constants. | Test Results |
| $ln ( \frac{{}^{206} {Pb}^{+}}{{}^{238} {U}^{16} {O}^{+}} ) = B \times ln ( \frac{{}^{238} {U}^{16} {O}_{2}^{+}}{{}^{238} {U}^{16} {O}^{+}} ) + ln ( A )$ | Equation (4), Transformed from Equation (3). | Transformation |
| $\frac{{( \frac{{}^{206} Pb}{{}^{238} U} )}_{Cstd 1}}{{( \frac{{}^{206} {Pb}^{+}}{{}^{238} {U}^{16} {O}^{+}} )}_{Cstd 1}} = \frac{{( \frac{{}^{206} Pb}{{}^{238} U} )}_{Cstd 2}}{{( \frac{{}^{206} {Pb}^{+}}{{}^{238} {U}^{16} {O}^{+}} )}_{Cstd 2}} = \frac{{( \frac{{}^{206} Pb}{{}^{238} U} )}_{Cun}}{{( \frac{{}^{206} {Pb}^{+}}{{}^{238} {U}^{16} {O}^{+}} )}_{Cun}} = T$ | Equation (5), $In case of {( \frac{{}^{238} {U}^{16} {O}_{2}^{+}}{{}^{238} {U}^{16} {O}^{+}} )}_{CStd 1} = {( \frac{{}^{238} {U}^{16} {O}_{2}^{+}}{{}^{238} {U}^{16} {O}^{+}} )}_{CStd 2} ,$ when measured under the same instrument conditions, the ratio of the lead-uranium mass ratio to the lead-uranium ion ratio of the | Test Results |
| | columbite-tantalite reference materials, the ratio of | |
| | the lead-uranium mass ratio to the lead-uranium ion | |
| | ratio of the sample to be tested, and the ratio of the | |
| | lead-uranium isotope ratio to the lead-uranium ion | |
| | ratio of the columbite-tantalite reference material | |
| | show the same variation relation, which can be | |
| | expressed by T. | |
| $\frac{{( \frac{{}^{206} Pb}{{}^{238} U} )}_{Cstd 2}}{{( \frac{{}^{206} Pb}{{}^{238} U} )}_{Cstd 1}} = \frac{{( \frac{{}^{206} {Pb}^{+}}{{}^{238} {U}^{16} {O}^{+}} )}_{Cstd 2}}{{( \frac{{}^{206} {Pb}^{+}}{{}^{238} {U}^{16} {O}^{+}} )}_{Cstd 1}} = \frac{{( \frac{{}^{206} {Pb}^{+}}{{}^{238} {U}^{16} {O}^{+}} )}_{Cstd 2}}{A \times {( \frac{{}^{238} {U}^{16} {O}_{2}^{+}}{{}^{238} {U}^{16} {O}^{+}} )}_{Cstd 2}^{B}}$ | Equation (6), which is obtained by merging and transforming Equation (3) and Equation (5). | Transformation |
| ${( \frac{{}^{206} Pb}{{}^{238} U} )}_{Cstd 2} = {( \frac{{}^{206} Pb}{{}^{238} U} )}_{Cstd 1} \times \frac{{( \frac{{}^{206} {Pb}^{+}}{{}^{238} {U}^{16} {O}^{+}} )}_{Cstd 2}}{A \times {( \frac{{}^{238} {U}^{16} {O}_{2}^{+}}{{}^{238} {U}^{16} {O}^{+}} )}_{Cstd 2}^{B}}$ | Equation (7), which is obtained by transforming Equation (6) | Transformation |
| ${( \frac{{}^{206} Pb}{{}^{238} U} )}_{Cun} = {( \frac{{}^{206} Pb}{{}^{238} U} )}_{Cstd 1} \times \frac{{( \frac{{}^{206} {Pb}^{+}}{{}^{238} {U}^{16} {O}^{+}} )}_{Cun}}{A \times {( \frac{{}^{238} {U}^{16} {O}_{2}^{+}}{{}^{238} {U}^{16} {O}^{+}} )}_{Cun}^{B}}$ | Equation (8), which is obtained by transforming Equation (6) | Transformation |
| Δt = Age_{UPbCStd2} − UPbt_{CStd2} | Equation (9), | Method |
| | which indicates a deviation value between the | defined in |
| | recommended age and the measured value, | the present |
| | i.e., a deviation value of the recommended value | disclosure |
| | of uranium-lead age of the columbite-tantalite | |
| | reference material 2 from the uranium-lead age | |
| | of the columbite-tantalite reference material 2 | |
| | calculated by this method. | |

An embodiment of the present disclosure provides a method for high-accuracy age determination of micron-scale columbite-tantalite. With two kinds of columbite-tantalite with known ages and different compositions as reference materials, the method includes: first determining a positive correlation between a lead-uranium ion ratio

$${( \frac{{}^{206} {Pb}^{+}}{{}^{238} {U}^{16} {O}^{+}} )}_{Cstd 1}$$

and a uranium ion ratio

$${( \frac{{}^{238} {U}^{16} {O}_{2}^{+}}{{}^{238} {U}^{16} {O}^{+}} )}_{Cstd 1}$$

of a reference material 1; calculating a lead-uranium isotope ratio

$${( \frac{{}^{206} Pb}{{}^{238} U} )}_{Cstd 2}$$

and measured age UPbt_{Cstd2} of a reference material 2 based on the same positive correlation; correcting an error to obtain a corrected positive correlation; and calculating a lead-uranium ratio

$${( \frac{{}^{206} Pb}{{}^{238} U} )}_{C u n}$$

and a lead-uranium age UPbt_{Cun} of a sample to be tested based on the corrected positive correlation and a lead-uranium iron ratio

$${( \frac{{}^{206} {Pb}^{+}}{{}^{238} {U}^{16} {O}^{+}} )}_{Cun}$$

and a uranium iron ratio

$${( \frac{{}^{238} {U}^{16} {O}_{2}^{+}}{{}^{238} {U}^{16} {O}^{+}} )}_{Cun}$$

of the sample to be tested.

The whole technical solution of the present disclosure will be explained in detail below by the specific embodiments.

### Embodiment 1: Preparation of Sample Mount Containing Two Columbite-Tantalite Reference Materials and One Columbite-Tantalite Sample to be Tested

The composition content of columbite-tantalite in nature varies, and the method of the present disclosure requires two columbite-tantalite reference materials with different compositions as calibration substances. Further, two kinds of columbite-tantalite with different compositions were selected as reference materials for instrument calibration in the present disclosure. The two reference materials included a columbite-tantalite reference material CStd1 (Nb₂O₅≈55.1%, Ta₂O₅≈24.2%, FeO≈13.0%, MnO≈57%, TiO₂≈0.5%, UPbt_{CStd1}≈380 Ma (million years)) from Fujian, China and a columbite-tantalite reference material CStd2 (Nb₂O₅≈48.0%, Ta₂O₅≈32.4%, FeO≈6.0%, MnO≈12.3%, TiO₂≈0.1%, UPbt_{CStd2}≈264 Ma (million years)) from Xinjiang, China. In this example of the present disclosure, CStd1 as the main reference material was used as an anchor sample in the double-reference-materials navigation method, and CStd2 as the secondary reference material was used as a target sample in the double-reference-materials navigation method. The sample to be tested was a columbite-tantalite sample Cun (Nb₂O₅≈13.1%, Ta₂O₅≈69.4%, FeO≈1.0%, MnO≈13.8%, TiO₂≈0.7%, Age_{UPbCun}≈140 Ma (million years)) from Hunan, China. Further, in this example of the present disclosure, this method was tested by using a columbite-tantalite sample Cun with the age known.

Referring to the requirements of the metrology technical specifications, the number of repeated measurements was selected as 10 for the test in a short period of time with the same instrument under the same test conditions, in order to check the repeatability of the test results. Generally, the particles of a columbite-tantalite reference material could be tested 0-2 times. Therefore, in order to ensure the number of tests, 7-10 particles of each of the above three columbite-tantalite samples were selected, respectively.

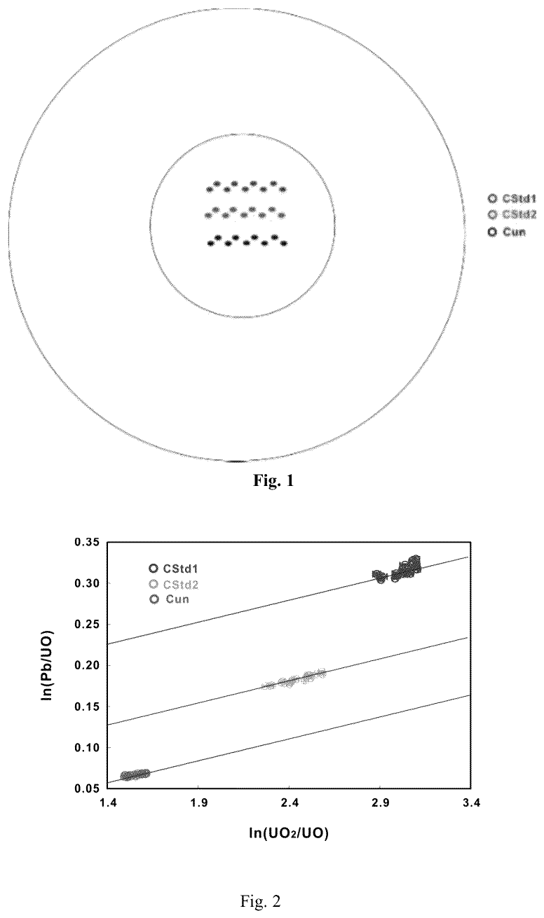

Specifically, the process of preparing a mount included: applying a double-sided adhesive tape of 10 cm*5 cm to a glass sheet of 10 cm*10 cm, and adhering 7-10 particles of the columbite-tantalite reference material CStd1 with the particle size of 100-150 microns, 7-10 particles of the columbite-tantalite reference material CStd2 with the particle size of 100-150 microns, and 7-10 particles of the sample Cun1 to be measured with the particle size of 100-150 microns onto the double-sided adhesive tape within a circle with a diameter of about 2 mm; mixing epoxy resin and a coagulant; placing a hollow polyethylene column with a smooth surface vertically on the double-sided adhesive tape, slowly pouring the vacuumized mixture of the epoxy resin and the coagulant along the inner surface of the hollow polyethylene column, performing vacuumization again and allowing standing to coagulate the mixture to obtain a coagulated columbite-tantalite reference material grains, i.e., a columbite-tantalite mount, that can be taken out from the hollow polyethylene column; and polishing the columbite-tantalite mount by sequentially using fine sandpaper and a polishing disk to expose the columbite-tantalite reference materials CStd1 and CStd2 and the sample Cun to be measured from a side of the mount, where the whole surface was clean and smooth. The prepared columbite-tantalite sample mount was shown in FIG. 1.

### Embodiment 2: Testing of Columbite-Tantalite with Secondary Ion Mass-Spectrometer

### 2.1 Washing the Sample Mount.

Specifically, in a first step, the surfaces of the samples were first washed with clear water; in a second step, the samples were placed in a beaker containing ethyl alcohol and ultrasonically washed for three minutes by an ultrasonoscope; and in a third step, the samples were dried for one hour in a drying oven.

2.2 Plating the Sample Mount with a Conductive Material.

Specifically, the exposed surface of the cleaned disc sample was plated with a continuous gold coating by using a Q150TE gold plating instrument of Quorum. In order to ensure the good conductivity of the sample, the thickness of the coating was 20 nm-50 nm, for example, 20 nm or 45 nm or the like.

### 2.3 Testing of Signals Required by Columbite-Tantalite by Using Secondary Ion Mass-spectrometry

Specifically, in a first step, an oxygen plasma ion source was focused onto the columbite-tantalite samples on the sample mount by means of Gaussian light to produce secondary ions of the columbite-tantalite samples; and secondary ions ²⁰⁴Pb⁺, ²⁰⁶Pb⁺, ²⁰⁷Pb⁺, ¹⁸¹Ta¹⁸O¹⁶O⁺, ¹⁸⁴W¹⁶O₂⁺, ²³⁸U⁺, ²³⁸U¹⁶O⁺, and ²³⁸U¹⁶O₂⁺ of the samples were allowed to sequentially pass through an electric field and a magnetic field to reach an ion signal detection system. A testing method was shown in Table 2 below.

TABLE 2

| | | Test time (s) of the method |
|---|---|---|
| Mass number | Ion type | of the present disclosure |
| 204.0 | ²⁰⁴Pb⁺ | 4.00 |
| 206.0 | ²⁰⁶Pb⁺ | 4.00 |
| 207.0 | ²⁰⁷Pb⁺ | 10.00 |
| 214.9 | ¹⁸¹Ta¹⁸O¹⁶O⁺ | 0.96 |
| 215.9 | ¹⁸⁴W¹⁶O₂⁺ | 0.96 |
| 238.1 | ²³⁸U⁺ | 6.00 |
| 254.0 | ²³⁸U¹⁶O⁺ | 4.00 |
| 270.0 | ²³⁸U¹⁶O₂⁺ | 2.96 |

### Embodiment 3: Calculating the Uranium-Lead Age UPbt_{Cun} of Sample to be Tested

According to the half-life principle

$$\frac{{}^{206} Pb}{{}^{238} U} = {e}^{{λ}_{238} * UPbt} - 1 , \qquad ( 1 )$$

the lead-uranium content ratio ²⁰⁶Pb/²³⁸U was required for calculation of the uranium-lead age UPbt using Equation

$$UPbt = ln ( ( \frac{{}^{206} Pb}{{}^{238} U} ) + 1 ) / {λ}_{238} . \qquad ( 2 )$$

Through several experiments, the inventor of the present disclosure found that three lead-uranium ion ratios could be measured by the instrument, namely,

$$\frac{{}^{206} {Pb}^{+}}{{238}^{} {U}^{+}} , \frac{{}^{206} {Pb}^{+}}{{}^{238} {U}^{16} {O}^{+}} , and \frac{{}^{206} {Pb}^{+}}{{}^{238} {U}^{16} {O}_{2}^{+}} .$$

The foregoing three lead-uranium ion ratios were unlike the real lead-uranium content ratio ²⁰⁶Pb/²³⁸U, which was due to the fact that when primary ions bombarded the sample surface, the yield of secondary ions ²⁰⁶Pb⁺ sputtered from the sample differed from the yields of three U-containing ions (i.e., ²³⁸U⁺, ²³⁸U¹⁶O⁺, and ²³⁸U¹⁶O₂⁺). Therefore, during uranium-lead dating of the columbite-tantalite using the secondary ion mass-spectrometer, it was necessary to correct the yields of uranium and lead to obtain the accurate lead-uranium age of the columbite-tantalite sample.

To solve this problem, the inventor of the present disclosure had the following findings based on researches.

First, after several experimental data analysis, a good positive correlation was found between the lead-uranium ion ratio

$$( \frac{{}^{206} {Pb}^{+}}{{}^{238} {U}^{16} {O}^{+}} )$$

and the uranium ion ratio

$$( \frac{{}^{238} {U}^{16} {O}_{2}^{+}}{{}^{238} {U}^{16} {O}^{+}} )$$

for the same columbite-tantalite, which could expressed in Equation (3) as (with A and B being constants):

$$\frac{{}^{206} {Pb}^{+}}{{}^{238} {U}^{16} {o}^{+}} = A \times {( \frac{{}^{238} {U}^{16} {o}_{2}^{+}}{{}^{238} {U}^{16} {o}^{+}} )}^{B} \qquad ( 3 )$$

Equation (3) was transformed to give the following Equation (4):

$$ln ( \frac{{}^{206} {Pb}^{+}}{{}^{238} {U}^{16} {o}^{+}} ) = B \times ln ( \frac{{}^{238} {U}^{16} {o}_{2}^{+}}{{}^{238} {U}^{16} {o}^{+}} ) + ln ( A ) \qquad ( 4 )$$

Based on the test results from multiple tests, fitting was performed to obtain results shown in FIG. 2, showing the positive correlation between the lead-uranium ion ratio

$$( \frac{{}^{206} {Pb}^{+}}{{}^{238} {U}^{16} {o}^{+}} )$$

and the uranium ion ratio

$$( \frac{{}^{238} {U}^{16} {o}_{2}^{+}}{{}^{238} {U}^{16} {o}^{+}} )$$

of columbite-tantalite of different ages. Here, the measurement results of three columbite-tantalite (including the columbite-tantalite reference materials CStd1 and CStd2, and the columbite-tantalite sample Cun to be measured). The measurements of the ratios of the three columbite-tantalite of different ages could be fitted into three straight lines, respectively.

Second, the inventor of the present disclosure also found through the tests that, measured under the same instrument conditions, when

$${( \frac{{}^{238} {U}^{16} {o}_{2}^{+}}{{}^{238} {U}^{16} {o}^{+}} )}_{CStd 1} = {( \frac{{}^{238} {U}^{16} {o}_{2}^{+}}{{}^{238} {U}^{16} {o}^{+}} )}_{CStd 2} ,$$

the following relation was present between the lead-uranium ion ratio and the lead-uranium mass ratio of the columbite-tantalite:

$$\frac{{( \frac{{}^{206} Pb}{{}^{238} U} )}_{Cstd 1}}{{( \frac{{}^{206} {Pb}^{+}}{{}^{238} {U}^{16} {o}^{+}} )}_{Cstd 1}} = \frac{{( \frac{{}^{206} Pb}{{}^{238} U} )}_{Cstd 2}}{{( \frac{{}^{206} {Pb}^{+}}{{}^{238} {U}^{16} {o}^{+}} )}_{Cstd 2}} = \frac{{( \frac{{}^{206} Pb}{{}^{238} U} )}_{Cun}}{{( \frac{{}^{206} {Pb}^{+}}{{}^{238} {U}^{16} {o}^{+}} )}_{Cun}} = T \qquad ( 5 )$$

That is, although the yields of lead and uranium from the same columbite-tantalite were different (i.e., T≠1) in the secondary ion mass spectrometer, the columbite-tantalite reference materials CStd1 and CStd2 and the sample Cun to be measured had the same yield of lead ²⁰⁶Pb as well as the same yield of ²³⁸U¹⁶O, when different columbite-tantalite was detected in the secondary ion mass spectrometer. Therefore, the ration between the lead-uranium isotope ratio and the lead-uranium ion ratio of the columbite-tantalite, i.e.,

$$\frac{( \frac{{}^{206} Pb}{{}^{238} U} )}{( \frac{{}^{206} {Pb}^{+}}{{}^{238} {U}^{16} {o}^{+}} )} ,$$

showed the same variation relation. This relation could be expressed using Equation (5) above.

Equation (3) and Equation (5) were merged and transformed into Equation (6) below.

$$\frac{{( \frac{{}^{206} Pb}{{}^{238} U} )}_{Cstd 2}}{{( \frac{{}^{206} Pb}{{}^{238} U} )}_{Cstd 1}} = \frac{{( \frac{{206}^{} {Pb}^{+}}{{}^{238} {U}^{16} {o}^{+}} )}_{Cstd 2}}{{( \frac{{}^{206} {Pb}^{+}}{{}^{238} {U}^{16} {o}^{+}} )}_{Cstd 1}} = \frac{{( \frac{{}^{206} {Pb}^{+}}{{}^{238} {U}^{16} {o}^{+}} )}_{Cstd 2}}{A \times {( \frac{{}^{238} {U}^{16} {o}_{2}^{+}}{{}^{238} {U}^{16} {o}^{+}} )}_{Cstd 2}^{B}} \qquad ( 6 )$$

According to the above experimental findings, the following calculation process was designed to establish a method for correction. That is, according to the two reference materials with known ages, the same slopes B of the two straight lines of reference materials could be obtained through iterative calculation, and then this fractionation law was applied to the sample Cun to be measured currently, such that the age UPbt_{Cun} of the sample could be obtained.

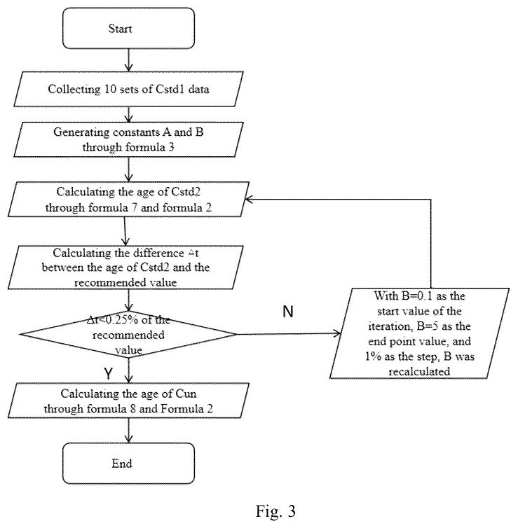

The specific determination process was as follows, with a flow chart shown in FIG. 3.

In step 1, the calibration curve of the reference material 1 was established based on the measurement data.

Through several experiments, the inventor of the present disclosure found a good positive correlation between the lead-uranium ion ratio

$$( \frac{{}^{206} {Pb}^{+}}{{}^{238} {U}^{16} {o}^{+}} )$$

and the uranium ion ratio

$$( \frac{{}^{238} {U}^{16} {o}_{2}^{+}}{{}^{238} {U}^{16} {o}^{+}} )$$

for the same columbite-tantalite, which could expressed in Equation (3) as (with A and B being constants):

$$\frac{{}^{206} {Pb}^{+}}{{}^{238} {U}^{16} {o}^{+}} = A \times {( \frac{{}^{238} {U}^{16} {o}_{2}^{+}}{{}^{238} {U}^{16} {o}^{+}} )}^{B} \qquad ( 3 )$$

Equation (3) was transformed to give the following Equation (4):

$$ln ( \frac{{}^{206} {Pb}^{+}}{{}^{238} {U}^{16} {o}^{+}} ) = B \times ln ( \frac{{}^{238} {U}^{16} {o}_{2}^{+}}{{}^{238} {U}^{16} {o}^{+}} ) + ln ( A ) \qquad ( 4 )$$

The columbite-tantalite reference material CStd1 with the age known was measured to obtain 10 data sets. The obtained measurement data sets

$$\frac{{}^{206} {Pb}^{+}}{{}^{238} {U}^{16} {o}^{+}}$$

were fitted with

$$\frac{{}^{238} {U}^{16} {o}_{2}^{+}}{{}^{238} {U}^{16} {o}^{+}}$$

according to Equation (4)

$$ln ( \frac{{}^{206} {Pb}^{+}}{{}^{238} {U}^{16} {o}^{+}} ) = B \times ln ( \frac{{}^{238} {U}^{16} {o}_{2}^{+}}{{}^{238} {U}^{16} {o}^{+}} ) + ln ( A )$$

to obtain the values of A and B.

In step 2, the lead-uranium isotope ratio of the reference material CStd2 was calculated based on the above calibration curve.

Through several experiments, the inventor of the present disclosure found

$${( \frac{{}^{238} {U}^{16} {{O}_{2}}^{+}}{{}^{238} U {}^{16} {O}^{-}} )}_{CStd 1} = {( \frac{{}^{238} {U}^{16} {{O}_{2}}^{+}}{{}^{238} U {}^{16} {O}^{+}} )}_{CStd 2} ,$$

which was combined with the calibration curve obtained in the above step to calculate the lead-uranium isotope ratio

$${( \frac{{}^{206} Pb}{{}^{238} U} )}_{Cstd 2}$$

of the columbite-tantalite reference material CStd2. First, the foregoing Equation 6 was transformed to obtain Equation 7 shown below.

$${( \frac{{}^{206} Pb}{{}^{238} U} )}_{Cstd 2} = {( \frac{{}^{206} Pb}{{}^{238} U} )}_{Cstd 1} \times \frac{{( \frac{{206}^{} {Pb}^{+}}{{}^{238} {U}^{16} {O}^{+}} )}_{Cstd 2}}{A \times {( \frac{{}^{238} {U}^{16} {{O}_{2}}^{+}}{{}^{238} U {}^{16} {O}^{+}} )}_{Cstd 2}^{B}} \qquad ( 7 )$$

The recommended value (0.0608, which was calculated according to the known age of 380 Ma and Equation (2) of

$${( \frac{{}^{206} Pb}{{}^{238} U} )}_{Cstd 1}$$

of the columbite-tantalite reference material Cstd1 and the above values of A and B were substituted into Equation (7), to thereby obtain the corrected lead-uranium ratio

$${( \frac{{}^{206} Pb}{{}^{238} U} )}_{Cstd 2}$$

of the columbite-tantalite reference material Cstd2 based on the currently obtained lead-uranium ion ratio

$${( \frac{{206}^{} {Pb}^{+}}{{}^{238} {U}^{16} {O}^{+}} )}_{Cstd 2}$$

and the uranium ion ratio

$${( \frac{{}^{238} {U}^{16} {{O}_{2}}^{+}}{{}^{238} U {}^{16} {O}^{-}} )}_{Cstd 2}$$

of the columbite-tantalite. The age UPbtCstd2 of the reference material Cstd2 was then calculated according to Equation (2)

$$UPb t = ln ( ( \frac{{206}^{} Pb}{{}^{238} U} ) + 1 ) / {λ}_{238} .$$

In step 3, a deviation value was calculated.

The deviation value of the recommended age from the measured value was calculated, i.e., the difference of the recommended value of uranium-lead age Age_{UPbCStd2} (264 Ma) of the columbite-tantalite reference material 2 from the uranium-lead age UPbt_{CStd2} of the columbite-tantalite reference material 2 as calculated by this method, Δt=Age_{UPbCStd2}−UPbt_{CStd2} (9).

In step 4, the deviation was corrected.

First, if it was determined that Δt was greater than 0.7 Ma, (264*0.25%=0.7, the deviation of the recommended value of uranium-lead age Age_{UPbCStd2} (264 Ma) of the columbite-tantalite reference material 2 was 0.25%, and the general recommended deviation of the columbite-tantalite reference material in the present disclosure was 0.25%. The slope value of B required to be recalculated with B=0.1 as the starting value of the iteration, B=5 as the end value, and a step of 1%. Each B value obtained was substituted into the steps 2 and 3 above, until Δt was less than or equal to 0.7 Ma.

In step 5, if Δt was less than or equal to 0.7 Ma, the above B could be used as the slope for the current measurement of the sample to be tested to proceed with the calculation of the age of the sample to be tested. That is, Equation 6 was transformed to obtain Equation 8 shown below.

$${( \frac{{}^{206} Pb}{{}^{238} U} )}_{Cun} = {( \frac{{}^{206} Pb}{{}^{238} U} )}_{Cstd 1} \times \frac{{( \frac{{206}^{} {Pb}^{+}}{{}^{238} {U}^{16} {O}^{+}} )}_{Cun}}{A \times {( \frac{{}^{238} {U}^{16} {{O}_{2}}^{+}}{{}^{238} U {}^{16} {O}^{+}} )}_{Cun}^{B}} \qquad ( 8 )$$

The recommended value (0.0608, which was calculated according to the known age of 380 Ma and Equation (2) of

$${( \frac{{}^{206} Pb}{{}^{238} U} )}_{Cstd 1}$$

of the columbite-tantalite reference material Cstd1 and the above values of A and B were substituted into Equation (8), to thereby obtain the corrected lead-uranium ratio

$${( \frac{{}^{206} Pb}{{}^{238} U} )}_{Cun}$$

of the columbite-tantalite sample Cun to be measured based on the currently obtained lead-uranium ion ratio

$${( \frac{{206}^{} {Pb}^{+}}{{}^{238} {U}^{16} {O}^{+}} )}_{Cun}$$

and the uranium ion ratio

$${( \frac{{}^{238} {U}^{16} {{O}_{2}}^{+}}{{}^{238} U {}^{16} {O}^{-}} )}_{Cun}$$

of the columbite-tantalite. Then, according to Equation (2)

$$UPb t = ln ( ( \frac{{206}^{} Pb}{{}^{238} U} ) + 1 ) / {λ}_{238} ,$$

the age value UPbt_{Cun} of the sample to be tested was calculated.

### Embodiment 4: Accuracy Analysis of Corrected U—Pb Age of Columbite-Tantalite with Different Ion Pairs

The inventor of the present disclosure found through experiments that the U—Pb age corrected by the ion pair

$$ln ( \frac{{}^{238} {U}^{16} {{O}_{2}}^{+}}{{}^{238} U {}^{16} {O}^{+}} ) vs ln ( \frac{{206}^{} {Pb}^{+}}{{}^{238} {U}^{16} {O}^{+}} )$$

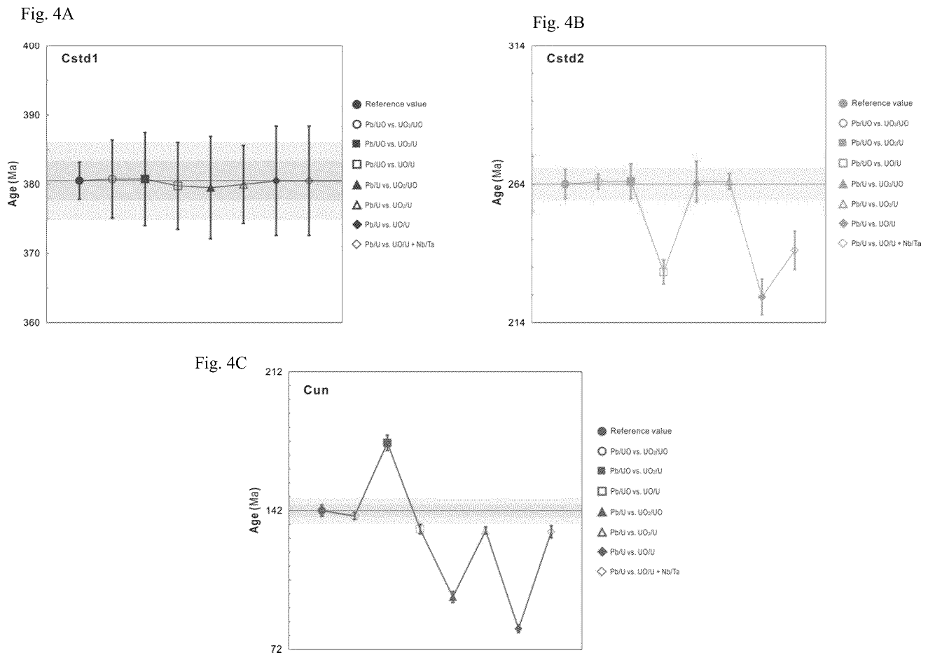

was most accurate. FIGS. 4A-4C showed a diagram of the results of U—Pb age corrected for columbite-tantalite with different ion pairs, and data were shown in Table 3. Here, a center point indicated an arithmetic mean of 20 analyses and the length of error bar indicated 1 sigma. in which FIG. 4A represents Cstd1, which is a columbite-tantalite reference material 1; FIG. 4B represents Cstd2, which is a columbite-tantalite reference material 2; and FIG. 4C represents Cun, which is an unknown columbite-tantalite sample to be tested.

TABLE 3
Mean Value of Resulting U—Pb Age of Columbite-
tantalite Corrected with Different Ion Pairs

| | Cstd1 | | Cstd2 | | Cun | |
|---|---|---|---|---|---|---|
| | Mean | Cstd1 | Mean | Cstd2 | Mean | Cun |
| | value | 1 sigma | value | 1 sigma | value | 1 sigma |
| | (million | deviation | (million | deviation | (million | deviation |
| | years) | % | years) | % | years) | % |
| Recommended value | 380.5 | 0.7 | 263.7 | 2.0 | 142.0 | 2.0 |
| Pb/UO vs. UO₂/UO | 380.7 | 1.5 | 264.9 | 1.0 | 139.3 | 1.3 |
| Pb/UO vs. UO₂/U | 380.7 | 1.8 | 265.0 | 2.4 | 176.2 | 2.2 |
| Pb/UO vs. UO/U | 379.7 | 1.7 | 232.2 | 1.9 | 132.6 | 1.8 |
| Pb/U vs. UO₂/UO | 379.5 | 1.9 | 264.9 | 2.8 | 98.5 | 2.8 |
| Pb/U vs. UO₂/U | 380.0 | 1.5 | 265.0 | 1.1 | 132.0 | 1.4 |
| Pb/U vs. UO/U | 380.5 | 2.1 | 223.3 | 2.9 | 82.4 | 2.3 |
| Pb/U vs. UO/U + | 380.5 | 2.1 | 240.1 | 2.9 | 131.3 | 2.3 |
| EPMA Nb—Ta | | | | | | |

In FIGS. 4A-4C, the solid circle represented the recommended value of the uranium-lead age. Based on the comparison of the SIMS measurements using different ion pairs, the smaller the deviation of the measurement result from the recommended value, the more accurate the result, and the more reliable the method. The hollow circle represented the uranium-lead age corrected with the ion pair

$$ln ( \frac{{}^{238} U {}^{16} {O}_{2}^{+}}{{}^{238} U {}^{16} {O}^{+}} ) vs ln ( \frac{{}^{206} Pb {}^{+}}{{}^{238} U {}^{16} {O}^{+}} ) ,$$

with which the uranium-lead age obtained was consistent with the recommended value within the range of derivation. The solid square represented the uranium-lead age corrected with the ion pair

$$ln ( \frac{{}^{238} U {}^{16} {O}_{2}^{+}}{{}^{238} U {}^{+}} ) vs ln ( \frac{{}^{206} Pb {}^{+}}{{}^{238} U {}^{16} {O}^{+}} ) ;$$

and with this ion pair, the uranium-lead ages of Cstd1 and Cstd2 could be made consist with the recommended value within the range of derivation by adjusting the slope, but the age of the sample to be tested was not consistent with the recommended value within the range of deviation. The hollow square represented the uranium-lead age corrected with the ion pair

$$ln ( \frac{{}^{238} U {}^{16} {O}^{+}}{{}^{238} U {}^{+}} ) vs ln ( \frac{{}^{206} Pb {}^{+}}{{}^{238} U {}^{16} {O}^{+}} ) ;$$

and with this ion pair, the uranium-lead ages of Cstd1 and Cstd2 could not be made consist with the recommended value within the range of derivation by adjusting the slope, and the age of the sample to be tested was not consistent with the recommended value within the range of deviation. The solid triangle represented the uranium-lead age corrected with the ion pair

$$ln ( \frac{{}^{238} {U}^{16} {O}_{2}^{+}}{{}^{238} U {}^{16} {O}^{+}} ) vs ln ( \frac{{}^{206} Pb {}^{+}}{{}^{238} U {}^{+}} ) ;$$

and with this ion pair, the uranium-lead ages of Cstd1 and Cstd2 could be made consist with the recommended value within the range of derivation by adjusting the slope, but the age of the sample to be tested was not consistent with the recommended value within the range of deviation. The hollow triangle represented the uranium-lead age corrected with the ion pair

$$ln ( \frac{{}^{238} {U}^{16} {O}_{2}^{+}}{{}^{238} U {}^{+}} ) vs ln ( \frac{{}^{206} Pb {}^{+}}{{}^{238} U {}^{+}} ) ;$$

and with this ion pair, the uranium-lead ages of Cstd1 and Cstd2 could be made consist with the recommended value within the range of derivation by adjusting the slope, but the age of the sample to be tested was not consistent with the recommended value within the range of deviation. The solid rhombus represented the uranium-lead age corrected with the ion pair

$$ln ( \frac{{}^{238} {U}^{16} {O}^{+}}{{}^{238} U {}^{+}} ) vs ln ( \frac{{}^{206} Pb {}^{+}}{{}^{238} U {}^{+}} ) ;$$

and with this ion pair, the uranium-lead ages of Cstd1 and Cstd2 could not be made consist with the recommended value within the range of derivation by adjusting the slope, and the age of the sample to be tested was not consistent with the recommended value within the range of deviation. The hollow rhombus represented the uranium-lead age obtained by using the method of Legros et al. (Reference 4), including correcting the SIMS measurement with the ion pair

$$ln ( \frac{{}^{238} {U}^{16} {O}^{+}}{{}^{238} U {}^{+}} ) vs ln ( \frac{{}^{206} Pb {}^{+}}{{}^{238} U {}^{+}} ) ;$$

to obtain a preliminary result, and then correcting the preliminary result with the Nb/Ta content ratio measured by EPMA to obtain the uranium-lead age. With this method, the uranium-lead ages of Cstd2 and the sample to be tested were made closer to the recommend value, but were not consistent with the recommended value within the range of deviation.

In summary, with the method recommended by the present disclosure, the uranium-lead age corrected by the ion pair

$$ln ( \frac{{}^{238} {U}^{16} {O}_{2}^{+}}{{}^{238} U {}^{16} {O}^{+}} ) vs ln ( \frac{{}^{206} Pb {}^{+}}{{}^{238} U {}^{16} {O}^{+}} ) ;$$

has the smallest deviation from the recommended value, showing the most accurate result and the most reliable method.

The embodiments described above only provide specific and detailed descriptions of several implementations of the present disclosure, and should not be construed to limit the patent scope of the present disclosure. It should be noted that several variations and improvements can be made by those of ordinary skills in the art without departing from the concept of the present disclosure, and these variations and improvements shall fall within the protection scope of the present disclosure. Therefore, the patent protection scope of the present disclosure shall be subjected to the accompanying claims.

What is claimed is:

1. A method for correcting an age of columbite-tantalite by using double-reference-materials, comprising: using two kinds of columbite-tantalite with known ages and different components as reference materials; determining a positive correlation between the lead-uranium ion ratio
$${( \frac{{}^{206} Pb {}^{+}}{{}^{238} U {}^{16} {O}^{+}} )}_{Cstd 1}$$
and the uranium ion ratio
$${( \frac{{}^{238} {U}^{16} {O}_{2}^{+}}{{}^{238} U {}^{16} {O}^{+}} )}_{Cstd 1}$$
of the reference material 1; calculating the lead-uranium isotope ratio
$${( \frac{{}^{206} Pb}{{}^{238} U} )}_{Cstd 2}$$
and the lead-uranium age UPbt_{Cstd2} of the reference material 2 based on the same positive correlation; calculating the lead-uranium ratio
$${( \frac{{}^{206} Pb}{{}^{238} U} )}_{Cun}$$
and the lead-uranium age UPbt_{Cun} of the sample to be tested based on the determined positive correlation and the lead-uranium iron ratio
$${( \frac{{}^{206} {Pb}^{+}}{{}^{238} U {}^{16} {O}^{+}} )}_{Cun}$$
and the uranium iron ratio
$${( \frac{{}^{238} {U}^{16} {O}_{2}^{+}}{{}^{238} U {}^{16} {O}^{+}} )}_{Cun}$$
of the sample; wherein the method further comprises:
S1, measuring the reference material 1 to obtain not less than 3 sets of lead-uranium ion ratio
$${( \frac{{}^{206} {Pb}^{+}}{{}^{238} U {}^{16} {O}^{+}} )}_{Cstd 1}$$
and the uranium ion ratio
$${( \frac{{}^{238} {U}^{16} {{O}_{2}}^{+}}{{}^{238} {U}^{16} {O}^{+}} )}_{Cstd 1} ,$$
and performing fitting according to the equation
$$ln ( \frac{{}^{206} {Pb}^{+}}{{}^{238} {U}^{16} {O}^{+}} ) = B \times ln ( \frac{{}^{238} {U}^{16} {{O}_{2}}^{+}}{{}^{238} {U}^{16} {O}^{+}} ) + ln ( A )$$
to obtain values of A and B;
S2, measuring the reference material 2 to obtain the lead-uranium ion ratio
$${( \frac{{}^{206} {Pb}^{+}}{{}^{238} {U}^{16} {O}^{+}} )}_{Cstd 2}$$
and the uranium ion ratio
$${( \frac{{}^{238} {U}^{16} {{O}_{2}}^{+}}{{}^{238} {U}^{16} {O}^{+}} )}_{Cstd 2}$$
of the reference material 2, substituting the values of A and B from S1 and the recommended value
$${( \frac{{}^{206} Pb}{{}^{238} U} )}_{Cstd 1}$$
of the reference material 1 into the equation
$${( \frac{{}^{206} Pb}{{}^{238} U} )}_{Cstd 2} = {( \frac{{}^{206} Pb}{{}^{238} U} )}_{Cstd 1} \times \frac{{( \frac{{}^{206} {Pb}^{+}}{{}^{238} {U}^{16} {O}^{+}} )}_{Cstd 2}}{A \times {( \frac{{}^{238} {U}^{16} {{O}_{2}}^{+}}{{}^{238} {U}^{16} {O}^{+}} )}_{Cstd 2}^{B}} ,$$
calculating the corrected lead-uranium ratio
$${( \frac{{}^{206} Pb}{{}^{238} U} )}_{Cstd 2}$$
of the reference material 2, and calculating measured age UPbt_{Cstd2} of the reference material 2 according to the equation as follows:
$$UPbt = ln ( ( \frac{{}^{206} Pb}{{}^{238} U} ) + 1 ) / {λ}_{238} ,$$
λ₂₃₈ is a decay constant, and λ₂₃₈=1.55125×10⁻¹⁰;
S3, calculating the deviation value of the recommended age from the measured age of the reference material 2 according to the equation Δt=Age_{UPbCStd2}−Upbt_{Cstd2};
S4, if a value of Δt is greater than Age_{UPbCStd2}×deviation of the reference material 2, recalculating a slope value of B with the above B=0.1 as a starting value of iteration, B=5 as an end value, and a step of 1%, substituting each value of B obtained into S2 and S3 to determine a value of B when Δt is less than or equal to the Age_{UPbCStd2}×deviation of the reference material 2, and if Δt is less than a value obtained by the Age_{UPbCStd2}×deviation of the reference material 2, directly going to S5; and
S5, measuring the lead-uranium ion ratio
$${( \frac{{}^{206} {Pb}^{+}}{{}^{238} {U}^{16} {O}^{+}} )}_{Cun}$$
and the uranium ion ratio
$${( \frac{{}^{238} {U}^{16} {{O}_{2}}^{+}}{{}^{238} {U}^{16} {O}^{+}} )}_{Cun}$$
of the sample, substitute the recommended value of
$${( \frac{{}^{206} Pb}{{}^{238} U} )}_{Cstd 1}$$
of the reference material Cstd1, the value of A, and the value of B obtained when Δt is less than or equal to the Age_{UPbCStd2}×deviation of the reference material 2 into the equation
$${( \frac{{}^{206} Pb}{{}^{238} U} )}_{Cun} = {( \frac{{}^{206} Pb}{{}^{238} U} )}_{Cstd 1} \times \frac{{( \frac{{}^{206} {Pb}^{+}}{{}^{238} {U}^{16} {O}^{+}} )}_{Cun}}{A \times {( \frac{{}^{238} {U}^{16} {{O}_{2}}^{+}}{{}^{238} {U}^{16} {O}^{+}} )}_{Cun}^{B}} ,$$
calculate the corrected lead-uranium ratio
$${( \frac{{}^{206} Pb}{{}^{238} U} )}_{Cun}$$
of the columbite-tantalite sample Cun, calculate the age value UPbt_{Cun} of the sample to be tested according to the equation
$$UPbt = ln ( ( \frac{{}^{206} Pb}{{}^{238} U} ) + 1 ) / {λ}_{238} .$$

2. The method of claim 1, further comprising: after obtaining the lead-uranium age UPbt_{Cstd2} of the reference material 2, the method further comprises performing deviation correction, determining a positive correlation after correction, and then calculating the lead-uranium ratio
$${( \frac{{}^{206} Pb}{{}^{238} U} )}_{Cun}$$
and the lead-uranium age UPbt_{Cun} of the sample.

3. The method of claim 1, wherein the two reference materials are columbite-tantalite reference material CStd1 and columbite-tantalite reference material CStd2.

4. The method of claim 1, wherein before measuring the samples, further comprising:
step 1, preparing a combined sample mount from the two reference material and a sample;
step 2, washing the sample mount; and
step 3, plating the sample mount with a conductive material.

5. The method of claim 4, wherein preparing a combined sample mount comprises:
step 1, applying a double-sided adhesive tape of 10 cm*5 cm to a glass sheet of 10 cm*10 cm, and adhering 7-10 particles of each of the reference material 1, the reference material 2, and the sample Cun1 with a particle size of 100-150 microns respectively onto the double-sided adhesive tape within a circle with a diameter of about 2 mm; and
step 2, placing a hollow polyethylene column with a smooth surface vertically on the double-sided adhesive tape, slowing pouring a vacuumized mixture of epoxy resin and a coagulant along an inner surface of the hollow polyethylene column, performing vacuumization again and allowing standing to coagulate the mixture to obtain a coagulated columbite-tantalite reference material grains, i.e., a columbite-tantalite mount, that can be taken out from the hollow polyethylene column, and polishing the columbite-tantalite mount by sequentially using fine sandpaper and a polishing disk to expose the reference material and the sample from a side of the mount.

6. The method of claim 4, wherein plating the sample mount with the conductive material comprises plating a surface of a cleaned disc at a side with exposed samples, with a continuous gold coating with a thickness of 20 nm-50 nm.

7. The method of claim 4, wherein measuring comprises measuring ion signals of the columbite-tantalite by using a secondary ion mass spectrometer SIMS.

8. The method of claim 7, wherein measuring the ion signals of the columbite-tantalite by using the secondary ion mass spectrometer (SIMS) comprises the steps of:
step 1, first, focusing an oxygen plasma ion source onto the columbite-tantalite samples on the sample mount by means of Gaussian light to produce secondary ions of the columbite-tantalite samples; and
step 2, allowing secondary ions ²⁰⁴Pb⁺, ²⁰⁶Pb⁺, ²⁰⁷Pb⁺, ¹⁸¹Ta¹⁸O¹⁶O⁺, ¹⁸⁴W¹⁶O₂⁺, ²³⁸U⁺, ²³⁸U¹⁶O⁺, and ²³⁸U¹⁶O₂⁺ of the samples to sequentially pass through an electric field and a magnetic field to reach an ion signal detection system, and performing detection to obtain a signal intensity of each of the secondary ions.
